(19) United States
(12) Patent Application Publication
(10) Pub. No.: US 2014/0044682 A1
(43) Pub. Date: Feb. 13, 2014

(54) GENERATION OF CARTILAGE EX VIVO FROM FIBROBLASTS

(71) Applicant: Pete O'Heeron, Houston, TX (US)

(72) Inventor: Pete O'Heeron, Houston, TX (US)

(21) Appl. No.: 13/962,241

(22) Filed: Aug. 8, 2013

Related U.S. Application Data
(60) Provisional application No. 61/681,731, filed on Aug. 10, 2012.

(51) Int. Cl. C12N 5/077

(52) U.S. Cl. CPC C12N 5/0655
USPC 424/93.7; 435/377

(57) ABSTRACT

Embodiments of the invention encompass the ex vivo production of cartilage from chondrocytes differentiated from fibroblasts or stem cells. In particular embodiments, fibroblasts are subjected to conditions to produce chondrocytes in the form of cartilage tissue, for example cartilage having a desired shape. In at least some embodiments, a mold for the desired shape of the cartilage is produced from imaging of a body region of an individual in need thereof, and the fibroblasts are seeded in the mold with particular conditions.

This application claims priority to U.S. Provisional Patent Application Ser. No. 61/681,731, filed Aug. 10, 2012, which is incorporated by reference herein in its entirety.

### TECHNICAL FIELD

The field of the present invention includes the fields of tissue engineering, medicine, surgery, anatomy, biology, cell biology and/or molecular biology. In certain embodiments the field of the invention concerns methods and compositions for treatment of medical conditions associated with body part(s) in need of cartilage.

### BACKGROUND OF THE INVENTION

Cartilage is a flexible connective tissue located in mammals in a variety of locations, including in joints between bones, the rib cage, the ear, the nose, the bronchial tubes and the intervertebral discs; it is a stiff material with less flexibility than muscle. Cartilage grows and repairs at a slower rate than other connective tissues, because cartilage does not contain blood vessels; instead, the chondrocytes are supplied by diffusion, helped by the pumping action generated by compression of the articular cartilage or flexion of the elastic cartilage. Furthermore, chondrocytes are bound in lacunae and cannot migrate to damaged areas, so cartilage damage is difficult to heal. The present invention provides solutions for needs in the art of cartilage repair.

### BRIEF SUMMARY OF THE INVENTION

The present invention is directed to methods and compositions for cartilage engineering to generate cartilage to an individual in need thereof. In specific embodiments, the invention concerns cells and tissues for the treatment of cartilage deficiencies. It is an exemplary object of the present invention to provide methods to repair or regenerate cartilage. The methods of the present invention generate cartilage of any kind, including elastic cartilage, hyaline cartilage and/or fibrocartilage, which differ in the relative amounts of its main components.

The present invention is directed to methods and compositions for treatment of an individual in need thereof, including treatment of an individual in need of cartilage repair. The present invention concerns methods and compositions for biological repair of any kind of cartilage. In particular aspects, the present invention concerns the fields of cartilage repair, including any kind of cartilage repair. More particularly, embodiments of the invention include methods for growing, proliferating, and/or differentiating cells into chondrocyte-like cells under mechanical stress for the production of cartilage ex vivo that is then placed in vivo in an individual. In particular aspects of the invention, cells utilized in the invention are subjected to mechanical strain, low oxygen (for example, <5%), or both for chondrogenic differentiation. In some embodiments, there is a method of differentiating human dermal fibroblasts into chondrocyte-like cells ex vivo.

Thus, in certain aspects, the invention generates natural tissue ex vivo, such as from fibroblasts, for example. More particularly, but not exclusively, the present invention relates to a method for growing and differentiating human fibroblasts into chondrocyte-like cells (or cells that function in the same capacity as chondrocytes), for example. The cells may be autologous or allogeneic or a mixture thereof, in certain embodiments.

In specific embodiments, the invention employs differentiation of certain cells into chondrocyte-like cells or cells that function in the same capacity as chondrocytes. In specific embodiments, human dermal fibroblasts (HDFs), for example, are differentiated into chondrocyte-like cells under particular conditions. Differentiation of cells into chondrocytes or chondrocyte-like cells may occur in any suitable manner, including ex vivo following procurement of fibroblasts, such as commercially or from a living individual or cell or tissue bank. Exemplary fibroblast cells may be harvested from skin, such as by a biopsy, for example. In some embodiments, the fibroblasts are obtained from the individual in need of cartilage.

In some embodiments of the invention, cartilage is imaged in an individual in need of cartilage repair or suspected of being in need of cartilage repair. Cartilage does not absorb x-rays under normal in vivo conditions, but a dye can be injected into the synovial joint that will cause the x-rays to be absorbed by the dye. The resulting void on the radiographic film between the bone and meniscus represents the cartilage. Other means of imaging cartilage is by magnetic resonance imaging (MRI). In embodiments of the invention, an image is taken of part of an individual to facilitate generation of cartilage tissue of a desired shape. In at least specific embodiments the image is three-dimensional. The imaging may be of any kind so long as it is suitable to allow generation of a desired cartilage shape. In specific embodiments, one could employ imaging, such as MRI or computed tomography (CT scan), of cartilage in a body location that is desired to be repaired or that is desired to be imaged to facilitate repair. For example, in cases where an ear or knee is in need of repair, one could take an image of a respective healthy ear or knee and produce an image (a minor image, in the case of the ear) of desired cartilage tissue of same.

An individual in need of cartilage repair may be of any kind so long as there is a detectable deficiency in cartilage tissue of any kind in the individual. In specific embodiments the cartilage deficiency comprises cartilage loss. An individual needing cartilage repair may be in need because of injury, disease, birth defect, environmental chemical exposure, a desire for cosmetic plastic surgery, excessive and/or substandard plastic surgery, the effects of obesity, sudden trauma, repetitive trauma, degeneration caused by wear and tear, the result of hip dysplasia, abusive use of drugs, allergic reactions, or a combination thereof. In cases where there is injury, the injury may be of any kind, including from combat, a fight, or sports, and/or immobility for extended periods of time, for example. The disease may be of any kind, including genetic, osteoarthritis, achondrogenesis, relapsing polychondritis, and so forth. The birth defect may be of any kind, such as microtia (including anotia), for example. An individual in need thereof may have a broken nose.

In certain aspects of the invention, the cells differentiate into chondrocyte cells or chondrocyte-like cells, such as wherein the chondrocyte cells or chondrocyte-like cells secrete a molecule selected from the group consisting of aggrecan, type II collagen, Sox-9 protein, cartilage link protein, perlecan, and combinations thereof. In particular cases, the cells are differentiated from fibroblast cells, and exemplary fibroblast cells include dermal fibroblasts, tendon fibroblasts, ligament fibroblasts, synovial fibroblasts, foreskin fibroblasts, or a mixture thereof.

In specific embodiments, there are no growth factors provided to the fibroblasts, including growth factors such as bone morphogenetic protein 2 (BMP-2), BMP-4, BMP-6, BMP-7, cartilage-derived morphogenetic protein (CDMP), transforming growth factor beta (TGF-β), insulin growth factor one (IGF-I), fibroblast growth factors (FGFs), basic fibroblast growth factor (bFGF), FGF-2, platelet-derived growth factor (PDGF), and a combination thereof. However, in alternative embodiments growth factors are employed in methods of the invention, such as provided to the fibroblasts, chondrocytes, and/or cartilage tissue, including BMP-2, BMP-4, BMP-6, BMP-7, CDMP, TGF-13, IGF-I, FGFs, bFGF, FGF-2, PDGF, and a combination thereof.

In some embodiments of the invention, there are methods and compositions related to delivering cartilage to a site in vivo in an individual in need thereof, wherein the cartilage was generated with a method of the invention. In specific embodiments, the delivery site is in vivo and in need of chondrocytes, including in need of cartilage. For example, a site in need of chondrocytes includes an ear, nose, knee, shoulder, elbow, and any other areas of the body where connective tissue is present or required. In some cases the cartilage is for a joint, whereas in other cases the cartilage is not for a joint.

In some embodiments, the fibroblasts are obtained from the individual in need of cartilage. In specific embodiments, resultant chondrocytes generated from fibroblasts are delivered to at least one location in an individual. In some cases, the fibroblasts are manipulated following being obtained, whether or not they are obtained from the individual in need thereof or whether or not they are obtained from a third party or commercially, for example. The fibroblasts may be expanded in culture. In certain embodiments, the fibroblasts are not provided growth factors, matrix molecules, mechanical strain, or a combination thereof, prior to or during or following implantation into the individual, although in alternative embodiments the fibroblasts are provided growth factors, matrix molecules, mechanical strain, or a combination thereof, prior to or during or following implantation into the individual.

Although the cartilage may be stored under suitable conditions for the individual from which the fibroblasts were derived, in some cases the cartilage is stored under suitable conditions for an individual from which the fibroblasts were not derived. The skilled artisan recognizes that in situations where the individual to which the cartilage is ultimately delivered is not the same individual that the original fibroblasts were obtained, one or more steps may be taken to prevent tissue rejection by the host body.

In some embodiments, there are both fibroblasts and chondrocytic cells in the cartilage. In some embodiments, the cartilage tissue is generated ex vivo but still retains one or more fibroblasts. Such tissue may still be delivered in vivo.

Thus, in specific embodiments one could generate high definition/resolution MRI or CT scan or other diagnostic imaging modality images of cartilage in the knee, shoulder, elbow, nose, ear, etc. In some embodiments, the MRI image would be utilized to generate a three-dimensional mold of the desired cartilage shape. In some embodiments, the mold is seeded with human dermal fibroblasts according to the present invention. Thus, the mold is subjected to conditions that facilitate generation of chondrocytes from fibroblasts, and in specific embodiments the conditions comprise low oxygen, mechanical stress, or any other atmospheric or biological condition(s) that may optimize differentiation of the fibroblasts into chondrocytes or chondrocyte-like cells, or a combination thereof. In specific embodiments, the fibroblasts to be differentiated to chondrocytes are exposed to a chamber that provides suitable conditions for chondrocyte differentiation. Within this environment, one can produce chondrocyte differentiation from fibroblasts and produce the cartilage tissue in the mold. Once the tissue is generated, it can be placed in the body at the appropriate location. In specific embodiments, at least one support is employed to support the cartilage; in specific embodiments the support is resorbable, although in some cases the support is not resorbable and is effectively permanent for the individual. In some cases, titanium, polymer, or another material is employed to support the cartilage.

In certain aspects of the invention, an individual is provided another therapy in addition to the methods of the invention. For example, before, during, and/or after delivery of the fibroblast cells, the individual may receive one or more antibiotics. Exemplary post-operative therapies includes Non Steroidal Anti-Inflammatory Drugs (NSAIDs), simple pain killers (analgesics), and/or muscle relaxants as needed, and it may be followed by a functional rehabilitation post-operatively, such as after the first, second, third or more post-operative week, for example. In specific embodiments, the individual may be provided one or more of an antibiotic, antifungal agent, or antiviral agent.

In a further embodiment, there is a kit comprising fibroblasts that are housed in one or more suitable containers. In specific embodiments, the kit further comprises one or more reagents suitable for enhancing ex vivo differentiation from fibroblasts to chondrocytes or chondrocyte-like cells. In some embodiments, the kit of the invention includes one or more apparatuses for delivery of cartilage to an individual. In some cases, the kit comprises one or more supports for stabilization of the cartilage upon in vivo delivery of the ex vivo-generated cartilage.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

The present invention incorporates by reference herein in its entirety U.S. patent application Ser. No. 12/775,720, filed May 7, 2010. The present invention incorporates by reference herein in its entirety U.S. patent application Ser. No. 61/557,479, filed Nov. 9, 2012.

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more elements or steps of the invention, for example. Some embodiments of the invention may consist of or consist essentially of one or more elements, method steps, and/or methods of the invention. It is contemplated that any method or composition described herein can be implemented with respect to any other method or composition described herein.

The term “chondrocyte-like cells” as used herein refers to cells that are not primary chondrocytes but are derived from fibroblasts, for example. These chondrocyte-like cells have a phenotype of chondrocytes (cells of cartilage) including a shape of chondrocytes (polygonal and/or rhomboidal cells, for example) and/or are able to aggregate and produce cartilage matrix components, such as sulfated proteoglycan and type II collagen, for example. Thus, exemplary markers of chondrocyte-like cells include one or more of aggrecan, which is a chondroitin sulfate and keratan sulfate proteoglycan, type II collagen, Sox-9 protein, cartilage link protein, and perlecan, which is a heparan sulfate proteoglycan, for example.

Although any tissues may be repaired at least in part by methods of the invention, including any cartilage tissues, in a particular exemplary embodiment, cartilage that is not in a joint or cartilage that is in a joint is repaired. A general embodiment of the invention is to use HDFs as cell sourcing for engineering new cartilage, because these cells are easy to harvest and to grow. The invention encompasses ex vivo differentiation of these cells into chondrocyte-like cells to produce a desired shape of cartilage tissue.

In specific embodiments, particular conditions are employed to facilitate differentiation of chondrocytes from fibroblasts ex vivo, including, for example, the following: 1) three dimensionality; 2) low oxygen tension; and 3) mechanical stress; 4) intermittent hydrostatic pressure; 5) fluid shear stress; and/or 6) other outside conditions that are conducive to chondrogenic differentiation.

In some embodiments, the fibroblast cells may be seeded in a matrix prior to and/or during chondrocyte differentiation and cartilage production. In embodiments wherein a matrix is employed (that may be referred to as a scaffold), the matrix may be comprised of a material that allows cells to attach to the surface of the material and form a three dimensional tissue. This material may be non-toxic, biocompatible, biodegradable, resorbable, or a combination thereof. In some embodiments, organic polymers such as polyglycolic acid (PGA), polylactic-co-glycolic acid (PLGA), poly-ε-caprolactone (PCL), polyamino acids, polyanhydrides, polyorthoesters; natural hydrogels such as collagen, hyaluronic acid, alginate, agarose, chitosan; synthetic hydrogels such as poly(ethylene oxide) (PEO), poly(vinyl alcohol) (PVA), poly(acrylic acid) (PAA), poly(propylene fumarate-co-ethylene glycol) [P(PF-co-EG) and copolymers thereof may be utilized. Alginate beads may be employed as the scaffold, in certain cases. In some embodiments, ceramic materials such as hydroxyapatite and/or tricalcium phosphate (TCP) may be used as the scaffolds in certain cases that require temporary or permanent structural support, for example. Collagen materials may be employed as the scaffold, in certain cases.

The cells may be put into a matrix made of one or more biopolymers, such as to mimic a natural matrix. The scaffold may be seeded in vitro or ex vivo, and in certain aspects growth factors are provided to the cells, the matrix, or both. The scaffold may be put into a chamber that may be a system for perfusion of medium and allows application of mechanical force to the scaffold and/or particular low oxygen conditions. Following delivery of the force, cells are assisted in differentiation, especially for generation of cartilage. In some embodiments, the matrix is employed with the cells in the mold (analogous to rebar for cement) and/or the matrix could be utilized with the fibroblast cells prior to the mold insertion.

In some aspects of the invention, the chondrocytes are generated and cartilage is produced in a chamber having particular conditions. The chamber may be capable of regulating one or more of the following parameters: temperature, medium pH, exchanges of gases, mechanical stimuli, pO₂, PCO₂, humidity, and nutrient diffusion, for example. A perfusion system may be present in the chamber, in specific embodiments, to provide constant supply of nutrients and to remove efficiently the waste products. One or more combinations of mechanical stresses may be provided, such as on an intermittent basis, including cell and tissue deformation, compressive and shear forces, fluid flow, and changes in hydrostatic pressure, for example. These conditions may be produced in the chamber, in certain aspects.

### I. Cells Utilized in the Invention

In certain embodiments of the invention, any cell may be employed so long as the cell is capable of differentiating into a chondrocyte or chondrocyte-like cell. However, in specific embodiments, the cell is a fibroblast cell, such as a dermal fibroblast, tendon fibroblast, ligament fibroblast, or synovial fibroblast, for example. Autologous cells may be utilized, although in alternative embodiments allogeneic cells are employed; in specific embodiments, the allogeneic cells have been assayed for disease and are considered suitable for human transmission. In certain aspects of the invention, the cell or cells are autologous, although in alternative embodiments the cells are allogeneic. In cases where the cells are not autologous, prior to use in the invention the cells may be processed by standard means in the art to remove potentially hazardous materials, pathogens, etc.

The rationale for using autologous HDFs as a means of cell sourcing follows from the following: 1) HDFs can be non-invasively harvested from a punch biopsy as little as a 3.0 mm diameter circular skin specimen, for example; 2) the risk of contamination from another donor (such as Hepatitis B Virus, Human Immunodeficiency Virus, Creutzfeldt-Jakob disease, etc.) does not exist; and 3) HDFs can expand easily in culture and differentiate into chondrocyte-like cells under particular culture conditions. Other fibroblast populations could be used, such as tendon or ligament, for example. In an embodiment, autologous fibroblasts are preferred. Some aspects of the invention may employ HDFs purchased commercially, such as from laboratories (such as Cascade Biologics). The cells can be adult HDFs or neonatal HDFs. Neonatal foreskin fibroblasts are a very convenient source of cells, for example. These cells are used commercially and are readily available and easy to grow.

In accordance with the invention, autologous HDFs are harvested from punch biopsy of skin tissue (6 mm) from the individual. In the laboratory, subcutaneous fat and deep dermis may be dissected away with scissors. The remaining tissue may be minced and incubated overnight in 0.25% trypsin at 4° C. Then, dermal and epidermal fragments may be separated, such as mechanically separated. The dermal fragments of the biopsy may be minced and the pieces may be used to initiate explant cultures. Fibroblasts harvested from the explants may be grown in Dulbecco's MEM (DMEM) with 10% calf serum at 37° C. in 8% CO₂. These cells may be expanded before being differentiated into chondrocytes, in particular aspects.

In particular aspects, chondrocyte-like differentiation of human dermal fibroblasts may be facilitated by employing mechanical strain. In specific embodiments of the invention, upon differentiation from fibroblasts, the resultant cells in vivo comprise expression of certain biochemical markers indicative of type I and II collagen and proteoglycans.

In particular aspects, chondrocyte-like differentiation of human dermal fibroblasts may occur in vivo, in which the micro-environment of the intervertebral disc is conducive for chondrocytic differentiation. Hydrostatic loading, hypoxia, cell to cell interaction with resident chondrocytic cells in the disc and other biochemical environments in the intervertebral disc may facilitate differentiation from fibroblast to chondrocytic cells, in particular embodiments. In specific embodiments of the invention, the cells in the intervertebral disc following cell transplantation will be a combination of fibrocytic and chondrocytic cells that produce both fibrous and chondrocytic tissues with biochemical markers of both type I and type II collagen and/or a number of proteoglycans found in cartilaginous and fibrous tissues.

II. Embodiments of Exemplary Methods of the Invention, including Methods of Repairing Damaged Cartilage

In embodiments of the invention, there are methods of differentiating cells, including fibroblasts (for example, human) into chondrocyte-like cells ex vivo. The methods may comprise the step of delivering fibroblasts to a mold for generation of a desired cartilage shape for an individual. The fibroblasts may be exposed to hypoxic conditions and/or mechanical strain prior to ex vivo production of the cartilage and delivery in vivo.

Mechanical stress/strain are important factors for chondrogenesis. The present method uses mechanical strains. In some embodiments, the method occurs in the presence of other types of pressure, including intermittent hydrostatic pressure, shear fluid stress, and so forth. In some embodiments, the method occurs in the absence or presence of low oxygen tension, growth factors, culturing in a matrix, and so forth.

Fibroblasts can be obtained from donor source (allogeneic) or autologous skin biopsy. Isolating cells from the skin and expanding them in culture may be employed, and in certain cases the cells are not manipulated or are minimally manipulated (for example, exposed to serum, antibiotics, etc).

In specific aspects of the invention, cells are induced to undergo differentiation into chondrocytes or chondrocyte-like cells. Such differentiation occurs prior to delivery in vivo. In specific embodiments of the invention, mechanical stress, low oxygen, or other conditions stimulate chondrogenic differentiation of HDFs.

In some methods of the invention, following obtaining of the fibroblast cells one may expand the number of cells, although in alternative embodiments fibroblasts are utilized for cartilage generation in the absence of any prior expansion. The skilled artisan recognizes that cells in culture require nutrition and one can feed the cells with media, such as FBS (fetal bovine serum). Contamination or infection may be prevented (for example, by adding antibiotics), in some cases. Prior to cartilage production, the cells may be washed with DMEM media to remove FBS and antibiotics, for example, and the cells may be used for cartilage production. The fluid suspension may contain a small amount of media including buffer, amino acids, salts, glucose and/or vitamins, for example. In vitro growth of the fibroblast cells may comprise at least one or more days for growth prior to use ex vivo for cartilage generation. In certain cases, the cells may be checked or monitored to ensure that at least some of the cells are dividing. Cells that are not dividing may be removed.

In embodiments of the invention, one obtains fibroblasts, for example from the individual being treated, obtains them from another individual (including a cadaver or living donor, for example), or obtains them commercially. One can take a skin biopsy and in some embodiments may manipulate the skin biopsy. For example, one can digest the skin tissue overnight to get fibroblasts, culture the cells to expand, and provide them to a system for cartilage production. Prior to delivery to the individual, the cells may be passaged one or more times depending on the number of cells needed, including 1, 2, 3, 4, 5, 6, 7, 8, 9, 10, or more times, for example. Passaging may occur over the course of one or more days, including 2, 3, 4, 5, 6, 7, 8, 9, or 10 days, or 1, 2, 3, 4, or more weeks, for example. In some embodiments, the cells are passaged for 5-7 days, for example.

### III. Support Embodiments

In some cases, cartilage generated by the methods of the invention is provided in vivo to an individual in conjunction with one or more supports for the cartilage. The support may be biodegradable or non-biodegradable and/or resorbable or non-resorbable, depending upon need. In cases where the support is resorbable, the support material may be of any kind in the art, including biopolymer. Lactide-based polymers including synthetic polyesters such as polylactide and copolymers with glycolide and ε-caprolactone are examples of resorbable polymers. In cases where the support is non-resorbable, the support material may be of any kind in the art, including metal or polymer. Non-resorbable polymers include polyacetal resins and/or polyetheretherketone. Slowly resorbable materials, such as ceramics and collagen, may be used for support.

Cartilage may be generated in vivo through an implantable reservoir or container used for the purpose of chondrogenic cell formation, and the reservoir can be removed after cartilage has formed, or the container may be made of absorbable materials that will be reabsorbed by the body during and after cartilage formation.

The support may be of any shape, including a shape that conforms to the shape of the cartilage, in some cases. The shape of the support may be a substantially identical shape of the support. In some cases, the support does not conform to the cartilage shape but is still supportive in function. Some support shapes include linear, round, tubular, rectangular, spherical, screw-like, conical, threaded, cup, box, and so forth.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Ex Vivo Production of Cartilage from Fibroblasts

An individual in need of cartilage or suspected of being in need of cartilage is subjected to method(s) of the invention. An individual in need of cartilage, such as having missing or defective cartilage, for example, is subjected to method(s) of the invention. In specific embodiments, an individual is diagnosed as being in need of cartilage. In some embodiments, the individual is not in need of vertebral disc repair.

Fibroblasts or stem cells from the individual are harvested, such as from the skin, for example, although in specific embodiments the fibroblasts or stem cells are obtained from another individual or commercially. The fibroblasts may be cultured after being obtained. The fibroblasts are subjected to conditions that facilitate chondrocyte differentiation, such as low oxygen, mechanical stress, or a combination thereof.

In some cases, the defective cartilage or a representative of the defective cartilage (such as a minor image of the defective cartilage, for example in a knee, shoulder, or ear) is imaged with appropriate methods, such as an MRI or CT scan, for example. The image is then employed to generate a mold of the desired shape of the defective cartilage. The fibroblasts are provided to the mold, and as the mold/fibroblasts are subjected to appropriate conditions, the fibroblasts differentiate into chondrocytes in the mold to produce cartilage tissue. In specific embodiments, however, the fibroblasts alone are subjected to appropriate conditions to produce chondrocytes prior to seeding in the mold, and in some cases the fibroblasts are subjected to appropriate conditions to produce chondrocytes prior to and following seeding in the mold. The mold itself may be able to generate the conditions necessary or the mold may be inserted into another container that generates those conditions.

The resultant cartilage is provided to an individual in need thereof, including the same individual from which the fibroblasts were harvested and/or to another individual in need of cartilage repair. In specific embodiments, the cartilage tissue is combined prior to or upon delivery with one or more supports to facilitate secure placement of the cartilage in its desired location, although in some cases a support is not needed. The support may be resorbable or may not be resorbable, depending on the desired location, thickness of the cartilage, and so forth.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of generating cartilage ex vivo, comprising the step of subjecting fibroblasts or stem cells to conditions to differentiate said fibroblasts or stem cells into chondrocytes ex vivo to produce cartilage.

2. The method of claim 1, wherein the cartilage is configured in the form of a desired shape.

3. The method of claim 1, wherein said conditions comprise low oxygen, mechanical stress, or a combination thereof.

4. The method of claim 2, wherein said desired shape is at least part of an ear.

5. The method of claim 2, wherein said desired shape is at least part of a nose.

6. The method of claim 2, further comprising the step of generating a mold of the desired shape.

7. The method of claim 1, further comprising the step of providing the cartilage to an individual that is in need of cartilage repair.

8. The method of claim 2, wherein said desired shape is utilized to replace or repair cartilage in one or more regions of the body of an individual, wherein said region requires connective tissue.

9. The method of claim 1, further comprising the step of imaging a part of the body of an individual that is in need of cartilage repair or that is suspected of being in need of cartilage repair.

10. The method of claim 1, further comprising the step of imaging a part of the body of an individual that is in need of cartilage repair and generating therefrom a mold of a desired shape of cartilage.

11. The method of claim 1, further comprising the step of imaging a part of the body of an individual wherein that part is not in need of repair and using that image to generate a mold for growth of cartilage to replace or repair an area in need of repair.

12. The method of claim 7, wherein the cartilage is provided to the individual with one or more supports.

13. The method of claim 12, wherein the support is resorbable.

14. The method of claim 12, wherein the support is comprised of a material that would be resorbed by the body of the individual during and/or after its function of cartilage formation is completed.

15. The method of claim 12, wherein the support is non-resorbable.

16. The method of claim 15, wherein the support is comprised of metal or one or more other materials that may remain in the body and act as a scaffolding to maintain shape and function of the cartilage.

17. The method of claim 7, wherein the cartilage tissue is delivered to a nose, ear, knee, shoulder, elbow or other area of the body where connective tissue is required for the individual.

18. The method of claim 7, wherein the cartilage tissue is not delivered to a joint.

19. The method of claim 7, wherein the cartilage tissue is not delivered to a vertebral disc.
